(19) United States
(12) Plant Patent Application Publication
(10) Pub. No.: US 2003/0101492 P1
(43) Pub. Date: May 29, 2003

(54) Sutera plant named Flicflac

(75) Inventor: Hubert Brandkamp, Isselburg-Anholt (DE)

Correspondence Address: WOOD, PHILLIPS, KATZ, CLARK & MORTIMER, 500 W. MADISON STREET, SUITE 3800, CHICAGO, IL, 60661, US

(73) Assignee: BRANDKAMP GmbH

(21) Appl. No.: 09/993,405

(22) Filed: Nov. 19, 2001

(51) Int. Cl. A01H 5/00

(52) U.S. Cl. PLT/263

(57) ABSTRACT

A new and distinct Sutera plant named Flicflac, characterized by its lavender flowers, Spreading, trailing habit, and dark green leaves.

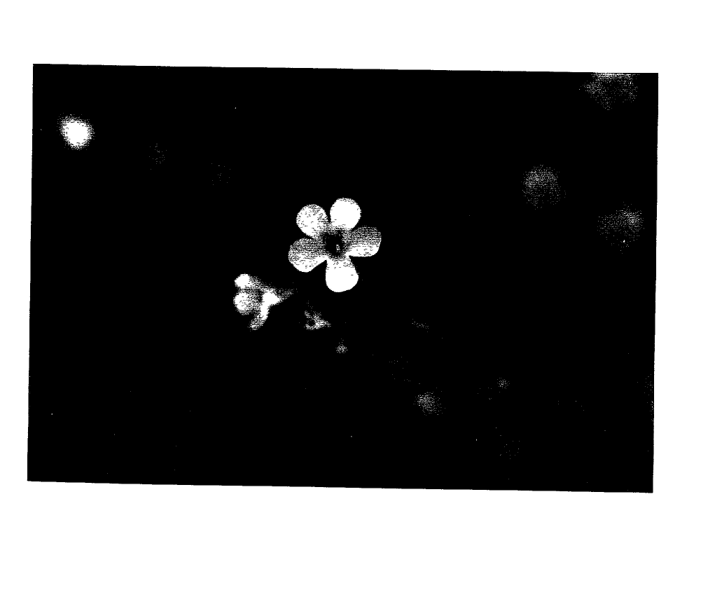

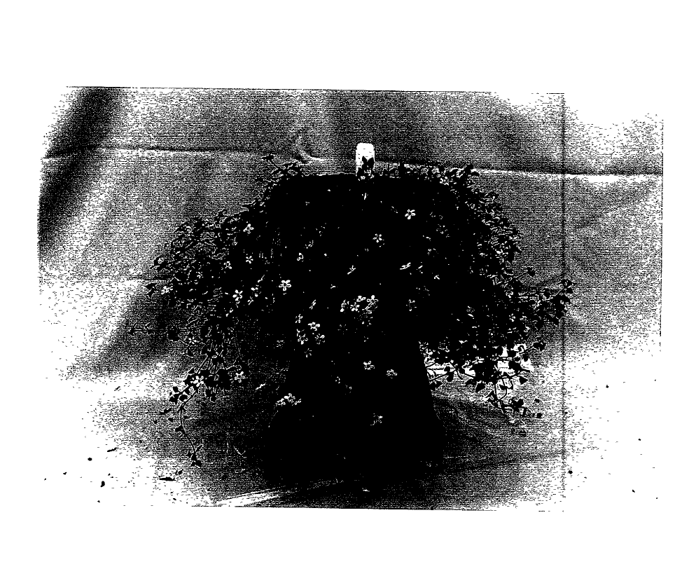

### BACKGROUND OF THE INVENTION

The present invention comprises a new and distinct Sutera plant, botanically known as Sutera cordata, and hereinafter referred to by the cultivar name Flicflac.

Flicflac originated as a naturally occurring mutation of the Sutera cordata cultivar Snowflake (not patented). The new cultivar was discovered by the inventor during 1999, in a controlled environment at lsselburg-Anholt, Germany.

Asexual reproduction of the new cultivar by terminal tip cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits pale lavender flowers,

(b) forms dark green foliage,

(c) exhibits a good basal branching character and

(c) exhibits a spreading and trailing growth habit.

The new cultivar of the present invention can be compared to ‘Lavender Storm’ (not patented). In side by side comparisons, plants of the new cultivar exhibit a more spreading growth habit, have longer internodes and larger flowers than ‘Lavender Storm’, as detailed in TABLE A, below.

TABLE A

| CHARACTERISTIC | FLICFLAC | SNOWFLAKE |
|---|---|---|
| Mature leaf length × width | 1.4 cm × 1.3 cm | 2 cm × 1.5 cm |
| Mature leaf color-upper | 147A | 137A |
| surface | | |
| Internode length | 1.5 cm | 2 cm |
| Plant diameter | 24.7 cm | 18.5 cm |

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown for 10 weeks in a greenhouse at West Chicago, Ill., U.S.A.

### DETAILED BOTANICAL DESCRIPTION

The cultivar Flicflac has not been observed under all possible environmental conditions to date. Accordingly, it is possible that the phenotype may vary somewhat with variations in the environment, such as temperature, light intensity, and day length. The chart used in the identification of colors described herein is the R.H.S. Colour Chart of The Royal Horticultural Society, London, England. The color values were determined on Aug. 15, 2001. The readings were taken between 1:00 and 3:00 p.m. under natural daylight conditions. The plants were produced from cuttings taken from stock plants and were grown in a double poly carbonate covered greenhouse under conditions comparable to those used in commercial practice while utilizing a soilless growth medium and maintaining temperatures of approximately 72° F. during the day and approximately 65° F. during the night. Plants used for the following descriptions and measurements were grown for 10 weeks from rooted cuttings.

Classification:

Botanical.—Sutera cordata cultivar Flicflac.

Parentage: Naturally occurring mutation of the Sutera cordata cultivar Snowflake (not patented).

Propagation:

Type cutting.—Terminal tip.

Time to initiate roots.—Approximately 7-10.

Time to develop roots.—Approximately 21 to 30 days.

Root description.—Fibrous, branching.

Plant Description:

Habit of growth.—Moderately vigorous with good basal branching. Pinching improves basal branching. A mature plant, 8 to 10 weeks after the planting of a rooted cutting, commonly measures approximately 6.6 cm in height and approximately 24.7 cm in diameter with an average of 5 main branches.

Form.—Spreading and trailing.

Stem.—Strong and woody. Approximately 1 mm in diameter, densely pubescent and 144A. Internode length at middle of branch is approximately 1.7 cm.

Foliage.—Leaves are non-fragrant, single, opposite, at an acute angle to the stem, and broadly elliptic with serrate margin, acute apex and attenuate base. Upper and lower surfaces are perberulent. Leaf length is approximately 1.4 cm and width is approximately 1.3 cm. Upper surface of young foliage is slightly darker than 143A, lower surface of young foliage is 147B. Upper surface of mature foliage is 147A, lower surface of mature foliage is 147B. Both upper and lower surfaces have pinnate venation closest to 144A. Petiole length is approximately 3 mm, diameter is approximately 1 mm, surface is very sparsely pubescent and color is closest to 144A.

Flowering Description:

Flowering habit.—Freely flowering.

Natural flowering season.—Year round in greenhouse environment; April through October in outdoor garden

Flower type.—Solitary, axillary, slaverform, sympetalous, actinomorphic. Flowers are persistent.

Peduncle.—Strong, densely pubescent with acute angle, approximately 9 mm in length, less than 1 mm in diameter and 144A.

Bud.—Tubular, approximately 7 mm in length, 3 mm in diameter. Bud, just before opening, is 188C at distil end with small area of 23B at center of tube.

Flower description.—Non-fragrant. Corolla is round, approximately 1.2 cm in diameter composed of five (5) flat lobes. Lobes have rounded tips, are approximately 5 mm long and 4 mm wide and have an entire margin. Color of upper surface of lobes when flower is opening is 85C, when fully opened it is 84D. Color of lower surface of lobes both when opening and when fully opened is 91C. Corolla tube is approximately 3 mm in diameter, 8 mm in length and pubescent on both inner and outer surface. Color of inner surface is 17A, outer surface is 32A near opening and 3D at base.

Calyx.—Five parted tube fused at base. Approximately 4 mm in length. Sepals are linear, less than 1 mm in width, have an acute apex, entire margin. The outer surface is densely pubescent and 143A.

Reproductive organs.—Androecium: There are 4 stamens—2 are 2 mm in length, 2 are 3 mm in length. Anthers are fan shaped, less than 1 mm in length. Pollen amount is moderate and color is 13A. Gynoecium: One pistil, 1.3 cm in length. Stigma shape is spatulate, color is 154B . Style length is 8 mm, color is 1D. Ovary diameter is 2 mm, color is 143A.

Seed production: Seed production has not been observed.

Disease resistance: Resistance to pathogens is not claimed

I claim:

1. A new and distinct cultivar of Sutera cordata named Flicflac substantially as herein shown and described, which:
(a) exhibits lavender flowers,
(b) medium green foliage,
(c) exhibits a good basal branching character and
(d) a spreading and trailing growth habit.
